(19) United States
(12) Patent Application Publication
(10) Pub. No.: US 2010/0312306 A1
(43) Pub. Date: Dec. 9, 2010

(54) Compression electric pulse tights

(76) Inventors: Aidan Walsh, Ridgefield, NJ (US); Alexander Wechsler, Paramus, NJ (US)

Correspondence Address: Aidan Walsh, 791 Norman Road, Ridgefield, NJ, 07657, US

(21) Appl. No.: 12/455,792

(22) Filed: Jun. 8, 2009

(51) Int. Cl. A61N 1/00; A41B 9/00

(52) U.S. Cl. 607/46; 607/149; 2/400

(57) ABSTRACT

This invention is a pair of Compression Tights that fit very snugly on the legs and stimulate the cells of the muscle via light, almost imperceptible, electric pulses. These tights can be worn during exercise to complement the benefits of exercise or after exercise to enhance the recovery process. Unlike current muscle stimulators, CEPT does not try to contract the muscle, instead it lightly stimulates the muscle. During use, an individual will notice a light tingle through the muscle. This device is not to be confused with stimulators designed to tone muscles, heal injuries, burn calories, or teach muscle memory.

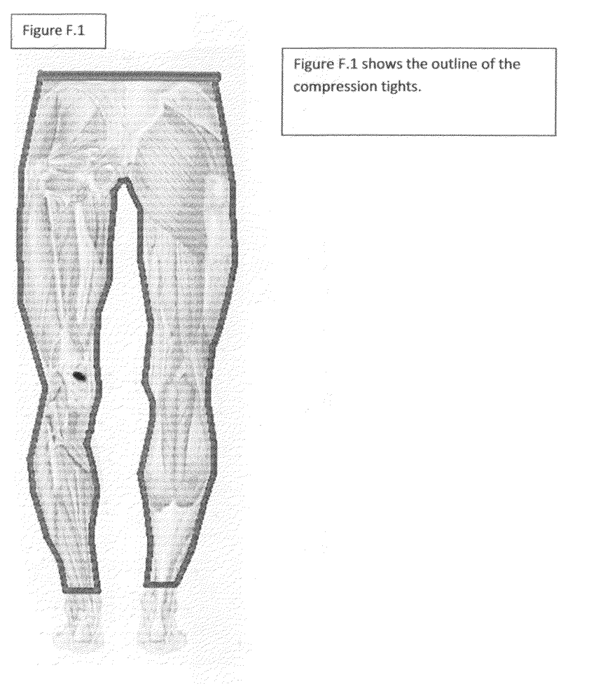

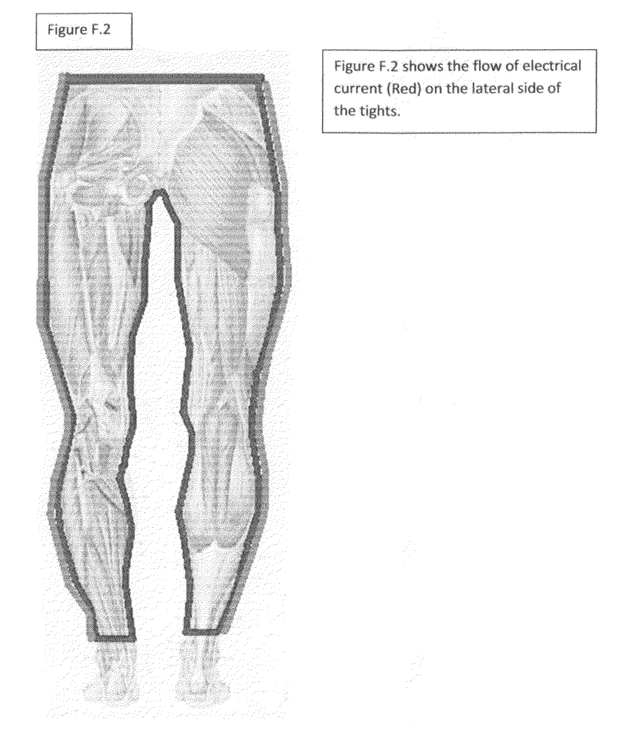

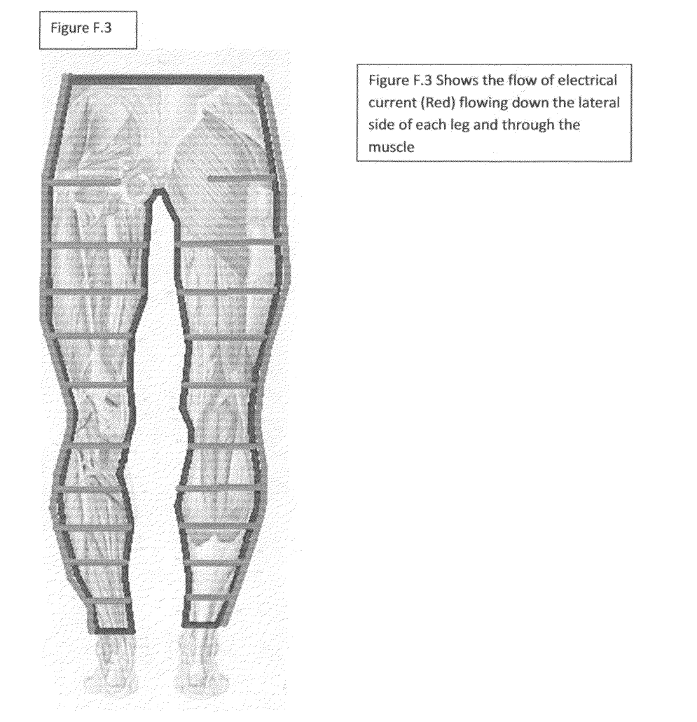

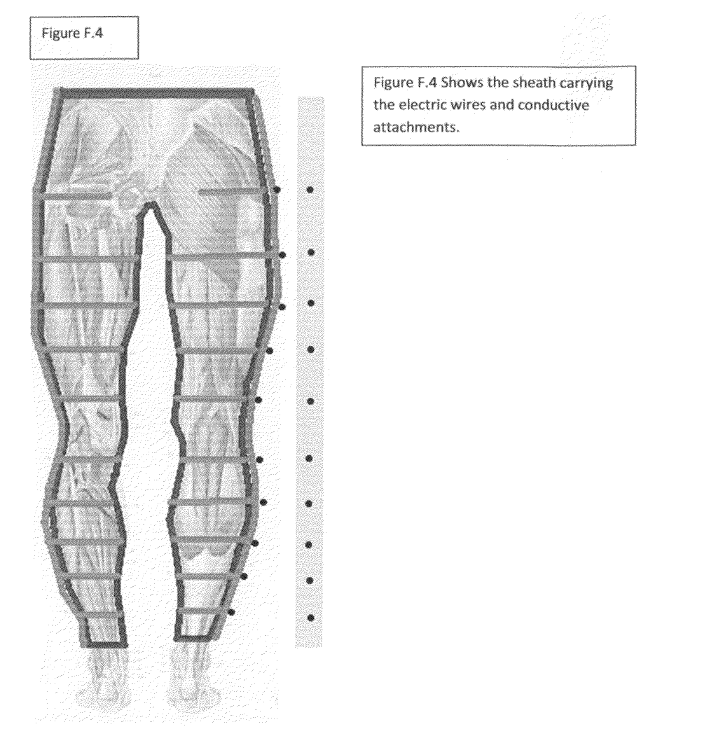

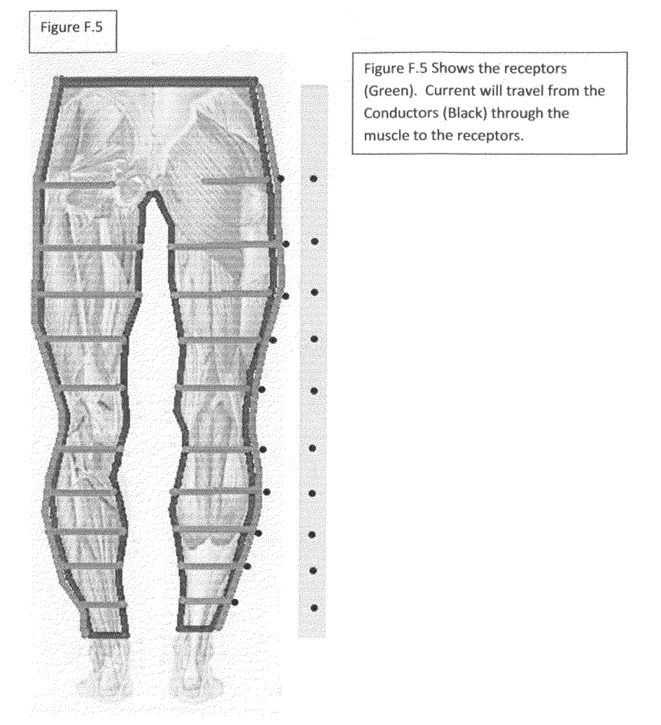

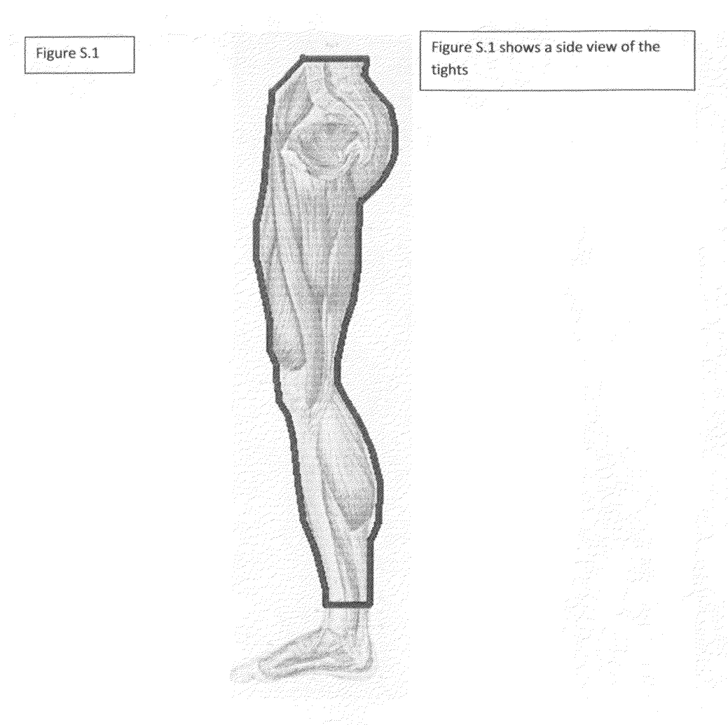

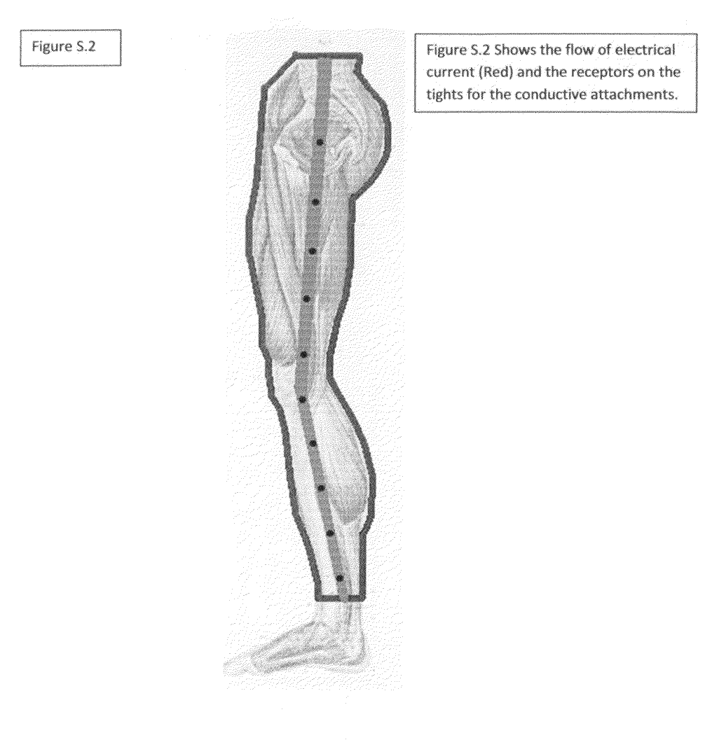

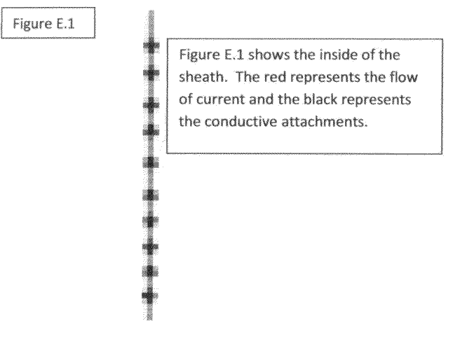

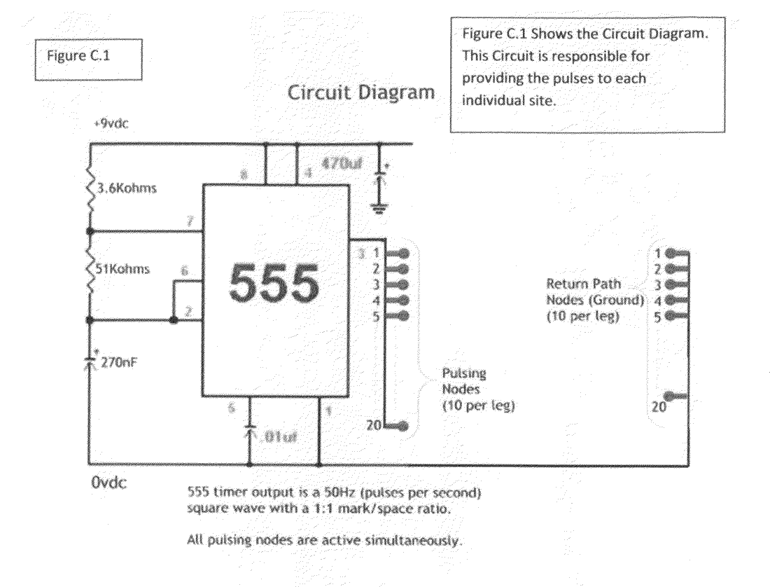

### FIELD OF INVENTION

This invention pertains to the complement of athletic training with regard to exercise and recovery.

### SPECIFICATION

The use of electric pulses has been previously used in the rehabilitation of injured muscles via electric muscle stimulator machines. Usage of electric pulse technology has previously been used in the industry of beauty products, as a means of toning muscles, burning calories, and most commonly shaping abdominal muscles. Typically, an individual would sit or relax while these stimulation machines contract the muscle. The CEPT takes those principles and for the first time instills these core fundamentals during activity to enhance basic exercise. The CEPT is also used post activity to encourage muscle recovery. The unique qualities that distinguish the CEPT from current muscle stimulators are 1) that the CEPT does not try to contract the muscle and 2) it can be worn during exercise.

As it stands in regards to the industry, there are many muscle stimulators that contract the muscle with a purpose focused on toning, repairing, and burning calories. This invention is unique because it stimulates the muscle during exercise. An individual can put on the CEPT prior to the commencement of exercise. As the individual is exercising, the muscles will receive light electric stimulation, which will complement the activity. The exercise in turn will become more effective and efficient. Current muscle stimulators have several large pads that send significant pulses through the muscle, thus contracting the muscle. The CEPT has an abundance of small pads that transmit a significantly lower level current through the muscle.

The human body is equipped with an auto-recovery response to damaged cells, which is fueled by a continuous flow of bioelectric current. During an engagement of athletic activity, an individual naturally tears fibers and damages cells. As previously stated, the body repairs these damaged cells by passing a bioelectric current through them. The CEPT would assist this form of healing.

The CEPT will simultaneously pass a current through each electrode at an imperceptible rate. An individual wearing the CEPT should notice a light, tingling sensation during each electric pulse, yet the muscle will not fully contract.

Each pulse will last 100th of 1 second and will repeat every one 50th of 1 second.

In the CEPT, the pulse would be passed through the muscle at no less than ten (10) sites on each leg. The pulse will travel from a conductor on the lateral side of each leg to a receiver on the medial side of each leg.

The CEPT will have many sites on each leg so as to stimulate as many cells as possible.

The intentional design for CEPT is for individuals to wear while participating in or recovering from activity. During exercise the CEPT can be worn to complement the stimulus of the activity. Once the activity is completed, the CEPT can be worn without significant physical restriction, thus allowing the individual to continue with their post activity routine.

The CEPT can be used for many different activities including, but not limited to, running, tennis, cycling, skiing, basketball, et cetera.

The structure of the CEPT will be such that the tights compress the muscle. The compression of muscle has been proven to increase blood flow and circulation to and from the muscle. The most common example of this can be seen in “flight socks”. People wear these tight knee high socks during a plane flight to prevent blood clotting on one's legs.

The CEPT will be made from a dry wicking material. Due to the fact that the CEPT can be worn during exercise, the material must be able to wick the sweat away from the person to increase the comfort level of the tights. The use of dry wicking fabrics has become standard in the exercise field.

The CEPT will be made of a flexible material. In order for the tights to effectively compress the muscle, the fabric must stretch and compress. If it were not flexible, then it would be very difficult to put on, and it would not conform to one's body. An example of this type of material would be Lycra.

The CEPT will be powered by a battery pack that attaches to the waist. This battery pack can be detached so that the tights are washable.

The battery pack will be attached to sheath. This sheath will encapsulate electric wires. The sheath will run from the hips to the ankles, on the lateral size of both legs and will attach to each individual site via conductive attachments. As with the battery pack, the sheath will be detachable so that the tights may be washed. Said battery pack and sheath will be water resistant to account for rain and excessive sweat.

The CEPT will be adjustable to suite various sizes and shapes. As peoples height and weight differ there will be a demand for various sizes. The CEPT will offer many options to cater to variations in body types.

As individuals differ in size and weight so too does the electrical resistance of the muscles. The CEPT will adjust accordingly to complete the circuit. For example a 200 pound man will have much more electrical resistance in their muscles than a 100 pound woman. With regard to the larger individual the CEPT must adjust the intensity of the pulse in order to complete the circuit and stimulate the muscles.

The CEPT can also be used in rehabilitation. It can be used to reduce muscle atrophy, to increase blood flow, to complement recovery, et cetera.

### BRIEF DESCRIPTION OF DRAWINGS

FIG. F1 Is a frontal view of the outline of the compression tights.

FIG. F2 is a frontal view of the tights depicting the flow of electrical current down the lateral side of each leg.

FIG. F3 Shows the flow of electrical current down the lateral side of each leg and into the muscles via ten conductive sites.

FIG. F4 Shows the sheath carrying the electric wires and the conductive attachments on the tights and sheath.

FIG. F5 Shows the receptors on the medial side of each leg. Current will travel from the waist down the lateral side of the leg, through the muscle, to the receptors on the medial side of each leg.

FIG. E1 Shows the inside of the sheath.

FIG. S1 Shows a side view of the tights.

FIG. S2 Shows the flow of electrical current and the receptors on the tights for the conductive attachments.

FIG. C1 Shows the Circuit Diagram. This diagram shows how the current will travel from the power supple to each individual pulsing node.

### SUMMARY OF INVENTION

Compression Electric Pulse Tights are a pair of compression tights that hold tightly to the muscle, while providing light electric pulses to stimulate cells during exercise and enhance recovery post exercise. The tights will be made of a Lycra type material that is dry wicking, breathable and flexible. The tights will have a very tight fit so as to compress the muscle. The tights will have no less than ten (10) electrode sites on each leg. These electrodes will pass an electric current from the lateral side of the leg to the medial side of the leg. The electrodes will be powered by a battery pack, which attaches to the waist of the tights. This battery pack will pass a current to each individual site via electric wires. The wires will be encapsulated in a detachable sheath that travels from the hip vicinity downward, passing through the upper and lower sections of the leg until ultimately reaching the ankle. The sheath will attach to each individual site via a conductive attachment. An example of a conductive attachment would be, but not limited to, snap on buttons or metal clips. The sheath and battery pack is removable so that the tights can be washed.

### DESCRIPTION OF THE INVENTION

A pair of Compression Electric Pulse Tights described as follows.
The CEPT have at least ten “sites” on each leg for a total of at least twenty “sites”. Each site will have an electrode that is designed to send an electronic pulse from the lateral side thru the muscle to the medial side. FIG. F.3 shows the transfer of current.
Each site is powered by individual wires contained in a sheath. This sheath will run down the lateral side of each leg and attach to each site via a conductive attachment. The sheath can be seen in FIG. E.1
The sheath is powered by a battery pack which attaches to the waist.
FIG. S.2 shows ten (10) sites. These ten sites are designed to cover most areas of the leg.
The tights will be made of a Lycra type material that is dry wicking, breathable and flexible while maintaining a compression on the muscle.
Each pair of CEPT's has at least twenty conductive sites, (ten on each leg). FIG. F.5
The sheath carrying the wires can be seen in FIG. E.1. This sheath will carry the electric current from the battery pack to each individual site
The sheath can be removed by unclipping or unhinging the conductive attachment so that the pants can be washed. FIG. F.4
The electric current will travel from the conductors on the lateral side of each leg to the receptors on the medial side of each leg. FIG. F.5
The electrodes in at each site will send a pulse that will last 100^{th} of 1 second and will repeat every one 50^{th of} 1 second.
The electrodes are built into the tights facing towards the skin at a minimum of ten sites in order to sufficiently stimulate the cells in the muscles
FIG. C.1 shows the Electrical Circuit that will be used to provide stimulation.

We claim that:

1- A pair of Compression Electric Pulse Tights (CEPT) FIG. F.5, worn during and after exercise, that pass the flow of electric current from a terminal to the leg muscles via multiple electrodes, said terminal attaches to the waste-band. The multitude of electrodes pass a light electric current from a conductor on the lateral side of each leg to a receptor on the medial side of each leg. Each electrode will be powered by an electronic wire which is encapsulated in a sheath. Said sheath has corresponding conductive attachments that transfer current from the wire to the electrodes. Said terminal and battery pack are attached to the waist.

2- The Tights in claim 1, wherein they can be worn during exercise.

3- The Tights in claim 1, wherein the goal is to lightly stimulate the muscle rather than contracting the muscle.

4- The Tights in claim 1, wherein there is a multitude of small electrodes. Said electrodes are built into the tights facing toward the skin.

5- The Tights in claim 1, wherein a terminal and battery pack provide the timing, and current.

6- The Tights in claim 1, wherein the terminal, battery pack, and sheath can be removed so that the tights can be washed. Said sheath can be attached or detached via conductive attachments.

7- The Tights in claim 1, wherein they can be adapted for most sports.

8- The Tights in claim 1, wherein they can be used for recovery.

9- The Tights in claim 1, wherein they can be used to increase circulation.

10- The Tights in claim 1, wherein they can be used for rehabilitation in physical therapy.

11- The Tights in claim 1, wherein they can be used by to reduce muscle atrophy.

12- The Tights in claim 1, wherein the material is dry wicking and flexible.

13- The Tights in claim 1, wherein they compress the muscle.

14- The Tights in claim 1, wherein they are water-resistant.

15- The Tights in claim 1, wherein the number of electrode sites can be increased as deemed necessary to provide optimal benefits.

16- The Tights in claim 1, wherein the principals can be improved or adjusted to maximize the quality of the product.

17- The Tights in claim 1, wherein they can be adjusted to contract the muscle depending on requirements.
